(19) United States
(12) Patent Application Publication
(10) Pub. No.: US 2013/0116270 A1
(43) Pub. Date: May 9, 2013

(54) NOVEL COMPOUNDS

(71) Applicants: Robert James Gleave, Cambridge (GB); Shuji Hachisu, Cambridge (GB); Lee William Page, Cambridge (GB)

(72) Inventors: Robert James Gleave, Cambridge (GB); Shuji Hachisu, Cambridge (GB); Lee William Page, Cambridge (GB)

(73) Assignee: CONVERGENCE PHARMACEUTICALS LIMTED

(21) Appl. No.: 13/696,793

(22) PCT Filed: May 10, 2011
(86) PCT No.: PCT/GB2011/050891

§ 371 Date: Jan. 18, 2013

(30) Foreign Application Priority Data
May 10, 2010 (GB) 1007791.5

(51) Int. Cl. C07D 471/10

(52) U.S. Cl. CPC C07D 471/10
USPC 514/278; 546/16

(57) ABSTRACT

The invention relates to novel spirocyclic derivatives with affinity for Ca.,2.2 calcium channels and which are capable of interfering with Caᵥ2.2 calcium channels; to processes for their preparation; to pharmaceutical compositions containing them; and to the use of such compounds in therapy for the treatment of pain.

The invention relates to novel spirocyclic derivatives with affinity for Caᵥ2.2 calcium channels and which are capable of interfering with Caᵥ2.2 calcium channels; to processes for their preparation; to pharmaceutical compositions containing them; and to the use of such compounds in therapy.

Presynaptic Caᵥ2.2 (N-type) voltage-gated calcium channels in the dorsal horn of the spinal cord modulate the release of key pro-nociceptive neurotransmitters such as glutamate, substance P (SP) and calcitonin-gene-related peptide (CGRP), indicating the potential therapeutic use of Caᵥ2.2 calcium channel blockers as analgesics.

Peptidic ω-conotoxins, isolated from the venom of cone snails, are selective for Caᵥ2.2 calcium channels and can block SP release in the spinal cord (Smith et al. (2002) Pain, 96: 119-127). Moreover, they are antinociceptive in animal models of chronic pain following intrathecal administration (Bowersox et al. (1996) Journal of Pharmacology and Experimental Therapeutics, 279: 1243-1249; Smith et al. (2002) supra), and are effective analgesics in clinical use, particularly in the treatment of neuropathic pain (Brose et al. (1997) Clinical Journal of Pain, 13: 256-259).

However, Caᵥ2.2 calcium channels are also important for normal neuronal function. Therefore, the aim is to identify novel molecules that preferentially block Caᵥ2.2 under conditions of increased neuronal excitability, so-called use-dependent blockers, as is the case in chronic pain syndromes (Winquist et al. (2005) Biochemical Pharmacology, 70: 489-499).

WO 2007/084314 (Incyte Corporation) discloses a series of cyclic compounds as modulators of 11-β hydroxyl steroid dehydrogenase type 1 which are claimed to be useful in disorders such as diabetes and obesity. WO 2005/047286 (Ono Pharm Co Ltd) discloses a series of heterocyclic spiro compounds as mitochondrial benzodiazepine receptor antagonists which are claimed to be useful for preventing and/or treating stress induced disorders. WO 99/65494 (Merck & Co Inc) discloses a series of spirocyclic compounds as prenyl-protein transferase inhibitors which are claimed to be useful in the treatment of cancer. WO 2006/006490 (Ono Pharm Co Ltd) discloses a series of spirocyclic compounds which are claimed to be useful in preventing and treating thrombosis, embolism, accompanying cerebrovascular diseases or venous vascular diseases.

The present invention provides compounds with affinity for Caᵥ2.2 calcium channels and which are capable of interfering with the affects of these channels. In a first aspect there is provided a compound of formula (I), or a salt thereof:

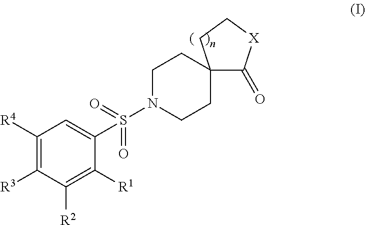

wherein R¹ and R⁴ are independently selected from hydrogen, chlorine, bromine, methyl, trifluoromethyl or trifluoromethoxy;
R² represents hydrogen, chlorine, fluorine, bromine, methyl, trifluoromethyl, difluoromethoxy or trifluoromethoxy;
R³ represents hydrogen, chlorine, bromine, trifluoromethyl or trifluoromethoxy; such that at least one of R¹, R², R³ and R⁴ represents a group other than hydrogen and such that when one of R¹, R², R³ or R⁴ represents methyl, at least one other of R¹, R², R³ or R⁴ represents a group other than hydrogen and such that when R² represents fluorine, R⁴ represents trifluoromethyl and such that when R³ represents trifluoromethyl, R² represents a group other than chlorine;
n represents an integer from 1 or 2;
X represents —N—(R⁵)—; and
R⁵ represents hydrogen or C₁₋₄ alkyl optionally substituted by one or more chlorine or fluorine atoms.

According to a particular aspect of the invention which may be mentioned there is provided a compound of formula (I), or a salt thereof:

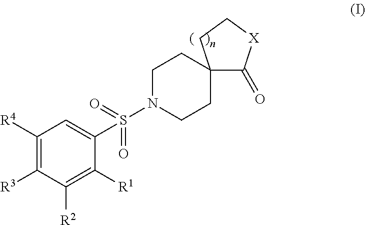

wherein R¹ and R⁴ are independently selected from hydrogen, chlorine, bromine, methyl, trifluoromethyl or trifluoromethoxy;
R² represents hydrogen, chlorine, fluorine, bromine, methyl, trifluoromethyl or trifluoromethoxy;
R³ represents hydrogen, chlorine, bromine, trifluoromethyl or trifluoromethoxy; such that at least one of R¹, R², R³ and R⁴ represents a group other than hydrogen and such that when one of R¹, R², R³ or R⁴ represents methyl, at least one other of R¹, R², R³ or R⁴ represents a group other than hydrogen and such that when R² represents fluorine, R⁴ represents trifluoromethyl and such that when R³ represents trifluoromethyl, R² represents a group other than chlorine;
n represents an integer from 1 or 2;
X represents —N—(R⁵)—; and
R⁵ represents hydrogen or C₁₋₄ alkyl optionally substituted by one or more chlorine or fluorine atoms.

As used herein, the term “alkyl” (when used as a group or as part of a group) refers to a straight or branched hydrocarbon chain containing the specified number of carbon atoms. For example, C₁₋₄ alkyl means a straight or branched hydrocarbon chain containing at least 1 and at most 4 carbon atoms. Examples of alkyl include, but are not limited to; methyl (Me), ethyl (Et), n-propyl, i-propyl and t-butyl.

In one embodiment, n represents 1. In an alternative embodiment, n represents 2.

In one embodiment, R¹ represents hydrogen, chlorine or methyl.

In one embodiment, R² represents hydrogen, fluorine, chlorine, methyl, trifluoromethyl or trifluoromethoxy.

In one embodiment, R³ represents hydrogen, trifluoromethyl or trifluoromethoxy.

In one embodiment, R⁴ represents hydrogen, chlorine or trifluoromethyl.

In one embodiment, R¹, R² and R⁴ each represent hydrogen and R³ represents trifluoromethyl or trifluoromethoxy. In a further embodiment, R¹, R² and R⁴ each represent hydrogen and R³ represents trifluoromethyl.

In one embodiment, R¹, R³ and R⁴ each represent hydrogen and R² represents trifluoromethyl, difluoromethoxy or trifluoromethoxy.

In one embodiment, R² and R⁴ each represent hydrogen, R¹ represents methyl or chlorine and R³ represents trifluoromethyl.

In one embodiment, R¹ and R³ each represent hydrogen, R² represents fluorine, chlorine, methyl or trifluoromethyl and R⁴ represents chlorine or trifluoromethyl.

In one embodiment, R¹ and R⁴ each represent hydrogen, R² represents chlorine and R³ represents trifluoromethoxy.

In one embodiment, R² and R³ each represent hydrogen, R¹ represents methyl and trifluoromethyl and R⁴ represents trifluoromethyl.

In one embodiment, R¹ and R³ each represent hydrogen, R² represents fluorine and R⁴ represents trifluoromethyl.

In one embodiment, one of R¹, R², R³ and R⁴ represents trifluoromethyl or trifluoromethoxy and the others all represent hydrogen. In a further embodiment, one of R¹, R², R³ and R⁴ represents trifluoromethyl and the others all represent hydrogen. In a yet further embodiment, R¹, R³ and R⁴ all represent hydrogen and R² represents trifluoromethyl or trifluoromethoxy. In an alternative yet further embodiment, R¹, R² and R⁴ all represent hydrogen and R³ represents trifluoromethyl.

In one embodiment, R⁵ represents hydrogen or C₁₋₄ alkyl (e.g. methyl or ethyl) optionally substituted by one or more chlorine or fluorine atoms (e.g. trifluoroethane). In a further embodiment, R⁵ represents hydrogen.

Particular compounds according to the invention include one or more compounds selected from:
8-{[2-Chloro-4-(trifluoromethyl)phenyl]sulfonyl}-2,8-diazaspiro[4.5]decan-1-one (E1);
9-{[4-(Trifluoromethyl)phenyl]sulfonyl}-2,9-diazaspiro[5.5]undecan-1-one (E2);
8-{[4-(Trifluoromethyl)phenyl]sulfonyl}-2,8-diazaspiro[4.5]decan-1-one (E3);
2-Methyl-8-{[4-(trifluoromethyl)phenyl]sulfonyl}-2,8-diazaspiro[4.5]decan-1-one (E4);
2-Methyl-8-{[3-(trifluoromethyl)phenyl]sulfonyl}-2,8-diazaspiro[4.5]decan-1-one (E5);
2-Methyl-8-({4-[(trifluoromethyl)oxy]phenyl}sulfonyl)-2,8-diazaspiro[4.5]decan-1-one (E6);
8-{[3-(Trifluoromethyl)phenyl]sulfonyl}-2,8-diazaspiro[4.5]decan-1-one (E7);
8-({4-[(Trifluoromethyl)oxy]phenyl}sulfonyl)-2,8-diazaspiro[4.5]decan-1-one (E8);
9-{[3-(Trifluoromethyl)phenyl]sulfonyl}-2,9-diazaspiro[5.5]undecan-1-one (E9);
9-({3-[(Trifluoromethyl)oxy]phenyl}sulfonyl)-2,9-diazaspiro[5.5]undecan-1-one (E10);
2-Ethyl-8-{[4-(trifluoromethyl)phenyl]sulfonyl}-2,8-diazaspiro[4.5]decan-1-one (E11);
2-Ethyl-8-{[3-(trifluoromethyl)phenyl]sulfonyl}-2,8-diazaspiro[4.5]decan-1-one (E12);
8-{[3-Fluoro-5-(trifluoromethyl)phenyl]sulfonyl}-2,8-diazaspiro[4.5]decan-1-one (E13);
9-({4-[(Trifluoromethyl)oxy]phenyl}sulfonyl)-2,9-diazaspiro[5.5]undecan-1-one (E14);
8-({3-[(Trifluoromethyl)oxy]phenyl}sulfonyl)-2,8-diazaspiro[4.5]decan-1-one (E15);
9-{[3-Fluoro-5-(trifluoromethyl)phenyl]sulfonyl}-2,9-diazaspiro[5.5]undecan-1-one (E16);
8-[(3,5-Dichlorophenyl)sulfonyl]-2,8-diazaspiro[4.5]decan-1-one (E17);
8-({3-Chloro-4-[(trifluoromethyl)oxy]phenyl}sulfonyl)-2,8-diazaspiro[4.5]decan-1-one (E18);
2-(2,2,2-Trifluoroethyl)-8-{[4-(trifluoromethyl)phenyl]sulfonyl}-2,8-diazaspiro[4.5]decan-1-one (E19);
8-{[3-Methyl-5-(trifluoromethyl)phenyl]sulfonyl}-2,8-diazaspiro[4.5]decan-1-one (E20);
8-{[2-Methyl-4-(trifluoromethyl)phenyl]sulfonyl}-2,8-diazaspiro[4.5]decan-1-one (E21);
8-{[2-Methyl-5-(trifluoromethyl)phenyl]sulfonyl}-2,8-diazaspiro[4.5]decan-1-one (E22);
8-{[3-Chloro-5-(trifluoromethyl)phenyl]sulfonyl}-2,8-diazaspiro[4.5]decan-1-one (E23);
9-({3-[(Difluoromethyl)oxy]phenyl}sulfonyl)-2,9-diazaspiro[5.5]undecan-1-one (E24);
8-{[3,5-Bis(trifluoromethyl)phenyl]sulfonyl}-2,8-diazaspiro[4.5]decan-1-one (E25);
8-({3-[(Difluoromethyl)oxy]phenyl}sulfonyl)-2,8-diazaspiro[4.5]decan-1-one (E26); and
9-{[2,5-Bis(trifluoromethyl)phenyl]sulfonyl}-2,9-diazaspiro[5.5]undecan-1-one (E27).

In one embodiment the compound of formula (I) is selected from:
8-{[4-(Trifluoromethyl)phenyl]sulfonyl}-2,8-diazaspiro[4.5]decan-1-one (E3); and
8-{[3-Fluoro-5-(trifluoromethyl)phenyl]sulfonyl}-2,8-diazaspiro[4.5]decan-1-one (E13).

In one embodiment the compound of formula (I) is selected from:
9-{[4-(Trifluoromethyl)phenyl]sulfonyl}-2,9-diazaspiro[5.5]undecan-1-one (E2);
8-{[4-(Trifluoromethyl)phenyl]sulfonyl}-2,8-diazaspiro[4.5]decan-1-one (E3);
2-Methyl-8-{[4-(trifluoromethyl)phenyl]sulfonyl}-2,8-diazaspiro[4.5]decan-1-one (E4);
9-{[3-(Trifluoromethyl)phenyl]sulfonyl}-2,9-diazaspiro[5.5]undecan-1-one (E9);
9-({3-[(Trifluoromethyl)oxy]phenyl}sulfonyl)-2,9-diazaspiro[5.5]undecan-1-one (E10);
2-Ethyl-8-{[4-(trifluoromethyl)phenyl]sulfonyl}-2,8-diazaspiro[4.5]decan-1-one (E11);
2-(2,2,2-Trifluoroethyl)-8-{[4-(trifluoromethyl)phenyl]sulfonyl}-2,8-diazaspiro[4.5]decan-1-one (E19); and
8-{[3-Methyl-5-(trifluoromethyl)phenyl]sulfonyl}-2,8-diazaspiro[4.5]decan-1-one (E20).

In a further embodiment the compound of formula (I) is selected from:
9-{[4-(Trifluoromethyl)phenyl]sulfonyl}-2,9-diazaspiro[5.5]undecan-1-one (E2);
8-{[4-(Trifluoromethyl)phenyl]sulfonyl}-2,8-diazaspiro[4.5]decan-1-one (E3);
2-Methyl-8-{[4-(trifluoromethyl)phenyl]sulfonyl}-2,8-diazaspiro[4.5]decan-1-one (E4);
2-Ethyl-8-{[4-(trifluoromethyl)phenyl]sulfonyl}-2,8-diazaspiro[4.5]decan-1-one (E11); and
2-(2,2,2-Trifluoroethyl)-8-{[4-(trifluoromethyl)phenyl]sulfonyl}-2,8-diazaspiro[4.5]decan-1-one (E19).

In a yet further embodiment the compound of formula (I) is:
8-{[4-(Trifluoromethyl)phenyl]sulfonyl}-2,8-diazaspiro[4.5]decan-1-one (E3).

Because of the potential use of compounds of formula (I) in medicine, salts of compounds of formula (I) are preferably pharmaceutically acceptable.

Certain compounds of formula (I) may in some circumstances form acid addition salts thereof. It will be appreciated that for use in medicine compounds of formula (I) may be used as salts, in which case the salts should be pharmaceutically acceptable.

Pharmaceutically acceptable salts include those described by Berge, Bighley and Monkhouse, J. Pharm. Sci., 1977, 66, 1-19. The term “pharmaceutically acceptable salts” includes salts prepared from pharmaceutically acceptable acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like.

Examples of pharmaceutically acceptable salts include those formed from maleic, fumaric, benzoic, ascorbic, pamoic, succinic, hydrochloric, sulfuric, bismethylenesalicylic, methanesulfonic, ethanedisulfonic, propionic, tartaric, salicylic, citric, gluconic, aspartic, stearic, palmitic, itaconic, glycolic, p-aminobenzoic, glutamic, benzenesulfonic, cyclohexylsulfamic, phosphoric and nitric acids.

It will be appreciated by those skilled in the art that certain protected derivatives of the compounds of formula (I), which may be made prior to a final deprotection stage, may not possess pharmacological activity as such, but may, in certain instances, be administered orally or parenterally and thereafter metabolised in the body to form compounds which are pharmacologically active. Such derivatives may therefore be described as “prodrugs”. All protected derivatives and prodrugs of compounds are included within the scope of the invention. Examples of suitable pro-drugs for the compounds of the present invention are described in Drugs of Today, Volume 19, Number 9, 1983, pp 499-538 and in Topics in Chemistry, Chapter 31, pp 306-316 and in “Design of Prodrugs” by H. Bundgaard, Elsevier, 1985, Chapter 1 (the disclosures in which documents are incorporated herein by reference). It will further be appreciated by those skilled in the art, that certain moieties, known to those skilled in the art as “pro-moieties”, for example as described by H. Bundgaard in “Design of Prodrugs” (the disclosure in which document is incorporated herein by reference) may be placed on appropriate functionalities when such functionalities are present within the compounds of formula (I). Therefore, in a further aspect, the invention provides a prodrug of a compound of formula (I).

It will be appreciated that certain compounds of formula (I), or their salts, may exist as solvates, such as hydrates. Where solvates exist, this invention includes within its scope stoichiometric and non-stoichiometric solvates.

It will be appreciated that certain compounds of formula (I), or their salts, may exist in more than one polymorphic form. The invention extends to all such forms whether in a pure polymorphic form or when admixed with any other material, such as another polymorphic form.

Certain compounds of formula (I) are capable of existing in stereoisomeric forms (e.g. diastereomers and enantiomers) and the invention extends to each of these stereoisomeric forms and to mixtures thereof including racemates. The different stereoisomeric forms may be separated one from the other by the usual methods, or any given isomer may be obtained by stereospecific or asymmetric synthesis. The invention also extends to any tautomeric forms and mixtures thereof.

The subject invention also includes isotopically-labeled compounds, which are identical to those recited in formula (I) and following, but for the fact that one or more atoms are replaced by an atom having an atomic mass or mass number different from the atomic mass or mass number most commonly found in nature. Examples of isotopes that can be incorporated into compounds of the invention include isotopes of hydrogen, carbon, nitrogen, fluorine such as ³H, ¹¹C, ¹⁴C and ¹⁸F.

Compounds of formula (I) and salts of said compounds that contain the aforementioned isotopes and/or other isotopes of other atoms are within the scope of the present invention. Isotopically-labeled compounds of the present invention, for example those into which radioactive isotopes such as ³H, ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. ¹¹C and ¹⁸F isotopes are particularly useful in PET (positron emission tomography). PET is useful in brain imaging. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of formula (I) and following of this invention can generally be prepared by carrying out the procedures disclosed in the Schemes and/or in the Examples below, by substituting a readily available isotopically labeled reagent for a non-isotopically labeled reagent. In one embodiment, the compounds of formula (I) or salts thereof are not isotopically labelled.

A compound of formula (I) may be prepared as set forth in the following Schemes and in the supporting compounds. The following processes form another aspect of the present invention.

The present invention also provides a process for the preparation of a compound of formula (I) or a salt thereof, which process comprises:

(a) reacting a compound of formula (II)

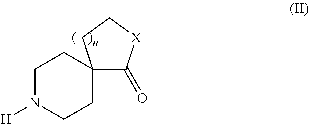

or a protected derivative thereof, wherein X and n are as defined above, with a compound of formula (III)

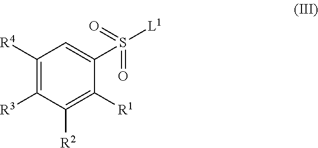

wherein R¹, R², R³ and R⁴ are as defined above and L¹ represents a suitable leaving group such as a halogen atom (e.g. chlorine);
(b) deprotecting a compound of formula (I) or converting groups which are protected; and optionally thereafter
(c) interconversion to other compounds of formula (I).

Process (a) typically comprises reaction of a compound of formula (II) with a compound of formula (III) in a suitable solvent, such as dichloromethane, in the presence of a base (for example triethylamine), at 0° C. to ambient temperature (for example ambient temperature).

In process (b), examples of protecting groups and the means for their removal can be found in T. W. Greene ‘Protective Groups in Organic Synthesis’ (J. Wiley and Sons, 3^{rd} Ed. 1999). Suitable amine protecting groups include sulfonyl (e.g. tosyl), acyl (e.g. acetyl, 2′,2′,2′-trichloroethoxycarbonyl, benzyloxycarbonyl or t-butoxycarbonyl) and arylalkyl (e.g. benzyl), which may be removed by hydrolysis (e.g. using an acid such as hydrochloric acid) or reductively (e.g. hydrogenolysis of a benzyl group or reductive removal of a 2′,2′,2′-trichloroethoxycarbonyl group using zinc in acetic acid) as appropriate. Other suitable amine protecting groups include trifluoroacetyl (—COCF₃) which may be removed by base catalysed hydrolysis or a solid phase resin bound benzyl group, such as a Merrifield resin bound 2,6-dimethoxybenzyl group (Ellman linker), which may be removed by acid catalysed hydrolysis, for example with trifluoroacetic acid.

Process (c) may be performed using conventional interconversion procedures such as epimerisation, oxidation, reduction, alkylation, nucleophilic or electrophilic aromatic substitution or amide bond formation. One such example of interconversion may be interconversion for a compound of formula (I) wherein R³ represents bromine to a compound of formula (I) wherein R³ represents cyano. Such interconversion may be carried out by treating the bromine compound with a cyanide salt (for example copper (I) cyanide) in a suitable solvent (such as N,N-dimethylformamide) at elevated temperatures (such as 200° C. using microwave irradiation). Alternatively the interconversion may be carried out using a cyanide salt (for example zinc cyanide) in the presence of a source of a palladium catalyst (for example tris(dibenzylideneacetone)dipalladium(0) and ligand (for example 1,1′-bis(diphenylphosphino)ferrocene) in a suitable solvent (such as N,N-dimethylformamide) at elevated temperatures (such as 120° C.). One example of an interconversion reaction includes reaction of a compound of formula (I) wherein one of R¹, R², R³ or R⁴ represents bromine to a compound of formula (I) wherein one of R¹, R², R³ or R⁴ represents methyl. Such interconversion comprises reaction in the presence of trimethylboroxine in the presence of a suitable base (such as potassium carbonate) and a suitable catalyst (such as Pd(PPh₃)₄) at elevated temperature (e.g. 100° C.). One further example of an interconversion reaction includes reaction of a compound wherein R⁵ represents hydrogen to a compound of formula (I) wherein R⁵ represents C₁₋₄ alkyl. Such interconversion comprises reaction of a compound of formula (I) wherein R⁵ represents hydrogen with a suitable alkylating agent (such as 2,2,2-trifluoroethyl trifluoromethanesulfonate) in a suitable solvent (such as tetrahydrofuran or dimethylformamide) in the presence of a suitable base (such as sodium hydride) at a temperature between 0° C. and ambient temperature.

Compounds of formula (II) and (III) are either commercially available, or may be prepared by known methods.

Compounds with affinity for Caᵥ2.2 calcium channels may be useful in the treatment or prophylaxis of pain, including acute pain, chronic pain, chronic articular pain, musculoskeletal pain, neuropathic pain, inflammatory pain, visceral pain, pain associated with cancer, pain associated with migraine, tension headache and cluster headaches, pain associated with functional bowel disorders, lower back and neck pain, pain associated with sprains and strains, sympathetically maintained pain; myositis, pain associated with influenza or other viral infections such as the common cold, pain associated with rheumatic fever, pain associated with myocardial ischemia, post operative pain, cancer chemotherapy, headache, toothache and dysmenorrhea.

‘Chronic articular pain’ conditions include rheumatoid arthritis, osteoarthritis, rheumatoid spondylitis, gouty arthritis and juvenile arthritis.

‘Pain associated with functional bowel disorders’ includes non-ulcer dyspepsia, non-cardiac chest pain and irritable bowel syndrome.

‘Neuropathic pain’ syndromes include: diabetic neuropathy, sciatica, non-specific lower back pain, trigeminal neuralgia, multiple sclerosis pain, fibromyalgia, HIV-related neuropathy, post-herpetic neuralgia, trigeminal neuralgia, and pain resulting from physical trauma, amputation, phantom limb syndrome, spinal surgery, cancer, toxins or chronic inflammatory conditions. In addition, neuropathic pain conditions include pain associated with normally non-painful sensations such as “pins and needles” (paraesthesias and dysesthesias), increased sensitivity to touch (hyperesthesia), painful sensation following innocuous stimulation (dynamic, static, thermal or cold allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia).

Other conditions which could potentially be treated by compounds of the present invention include neurodegenerative diseases and neurodegeneration, neurodegeneration following trauma, tinnitus, dependence on a dependence-inducing agent such as opiods (e.g. morphine), CNS depressants (e.g. ethanol), psychostimulants (e.g. cocaine) and nicotine.

Neurodegenerative diseases include dementia, particularly degenerative dementia (including senile dementia, dementia with Lewy bodies, Alzheimer's disease, Pick's disease, Huntingdon's chorea, Parkinson's disease and Creutzfeldt-Jakob disease, ALS, motor neuron disease); vascular dementia (including multi-infarct dementia); as well as dementia associated with intracranial space occupying lesions; trauma; infections and related conditions (including HIV infection, meningitis and shingles); metabolism; toxins; anoxia and vitamin deficiency; and mild cognitive impairment associated with ageing, particularly Age Associated Memory Impairment.

The compounds of formula (I) may also be useful for neuroprotection and in the treatment or prophylaxis of neurodegeneration following trauma such as stroke, cardiac arrest, pulmonary bypass, traumatic brain injury, spinal cord injury or the like.

Another condition which could potentially be treated by compounds of formula (I) is spasticity or muscular hypertonicity.

Thus, according to one aspect of the invention, there is provided a compound of formula (I) as defined herein for use in therapy.

In one embodiment, the therapy is to the treatment or prophylaxis of any of the disorders described herein, in particular pain. In one particular embodiment, the therapy is to the treatment of any of the disorders described herein, in particular pain.

According to a further aspect, there is provided a use of a compound of formula (I), or a pharmaceutically acceptable salt thereof, in the manufacture of a medicament for the treatment or prophylaxis of any of the disorders herein, in particular pain. More particularly, there is provided a use of a compound of formula (I), or a pharmaceutically acceptable salt thereof, in the manufacture of a medicament for the treatment of any of the disorders herein.

According to another aspect, there is provided a method of treatment of any of the disorders herein, in particular pain in humans, which method comprises the administration to the human in need of such treatment, an effective amount of a compound of formula (I), or a pharmaceutically acceptable salt thereof.

In the context of the present invention, treatment refers to symptomatic treatment.

In order to use a compound of formula (I), or a pharmaceutically acceptable salt thereof, for the treatment or prophylaxis of humans and other mammals, it is normally formulated in accordance with standard pharmaceutical practice as a pharmaceutical composition. Therefore in another aspect of the invention there is provided a pharmaceutical composition comprising a compound of formula (I), or a pharmaceutically acceptable salt thereof, adapted for use in human or veterinary medicine.

In order to use the compounds of formula (I) in therapy, they will normally be formulated into a pharmaceutical composition in accordance with standard pharmaceutical practice. The present invention also provides a pharmaceutical composition, which comprises a compound of formula (I), or a pharmaceutically acceptable salt thereof, and optionally a pharmaceutically acceptable excipient.

When used in the treatment or prophylaxis of pain, the compound of formula (I) or a pharmaceutically acceptable salt thereof may be used in combination with other medicaments indicated to be useful in the treatment or prophylaxis of pain of neuropathic origin including neuralgias, neuritis and back pain, and inflammatory pain including osteoarthritis, rheumatoid arthritis, acute inflammatory pain, back pain and migraine. Such therapeutic agents include for example COX-2 (cyclooxygenase-2) inhibitors, such as celecoxib, deracoxib, rofecoxib, valdecoxib, parecoxib, COX-189 or 2-(4-ethoxy-phenyl)-3-(4-methanesulfonyl-phenyl)-pyrazolo[1,5-b]pyridazine (WO99/012930); 5-lipoxygenase inhibitors; NSAIDs (non-steroidal anti-inflammatory drugs) such as diclofenac, indomethacin, nabumetone or ibuprofen; bisphosphonates, leukotriene receptor antagonists; DMARDs (disease modifying anti-rheumatic drugs) such as methotrexate; adenosine A1 receptor agonists; sodium channel blockers, such as lamotrigine; NMDA (N-methyl-D-aspartate) receptor modulators, such as glycine receptor antagonists or memantine; ligands for the α₂δ-subunit of voltage gated calcium channels, such as gabapentin and pregabalin; tricyclic antidepressants such as amitriptyline; neurone stabilising antiepileptic drugs; cholinesterase inhibitors such as galantamine; mono-aminergic uptake inhibitors such as venlafaxine; opioid analgesics; local anaesthetics; 5HT₁ agonists, such as triptans, for example sumatriptan, naratriptan, zolmitriptan, eletriptan, frovatriptan, almotriptan or rizatriptan; nicotinic acetyl choline (nACh) receptor modulators; glutamate receptor modulators, for example modulators of the NR2B subtype; EP₄ receptor ligands; EP₂ receptor ligands; EP₃ receptor ligands; EP₄ agonists and EP₂ agonists; EP₄ antagonists; EP₂ antagonists and EP₃ antagonists; cannabinoid receptor ligands; bradykinin receptor ligands; vanilloid receptor or Transient Receptor Potential (TRP) ligands; and purinergic receptor ligands, including antagonists at P2X₃, P2X_{2/3}, P2X₄, P2X₇ or P2X_{4/7}. Additional COX-2 inhibitors are disclosed in U.S. Pat. No. 5,474,995, U.S. Pat. No. 5,633,272; U.S. Pat. No. 5,466,823, U.S. Pat. No. 6,310,099 and U.S. Pat. No. 6,291,523; and in WO 96/25405, WO 97/38986, WO 98/03484, WO 97/14691, WO99/12930, WO00/26216, WO00/52008, WO00/38311, WO01/58881 and WO02/18374.

When used in the treatment or prophylaxis of Alzheimer's disease, the compound of formula (I) or a pharmaceutically acceptable salt thereof may be used in combination with other medicaments indicated to be useful as either disease modifying or symptomatic treatments of Alzheimer's disease.

Suitable examples of such other therapeutic agents may be agents known to modify cholinergic transmission such as 5-HT_{1A} antagonists, (e.g. lecozotan), 5-HT6 antagonists, M1 muscarinic agonists, M2 muscarinic antagonist, acetylcholinesterase inhibitors (e.g tetrahydroaminoacridine, donepezil or rivastigmine), or allosteric modulators, nicotinic receptor agonists or allosteric modulators, symptomatic agents such as 5-HT6 receptor antagonists, e.g. SB742457, H3 receptor antagonists e.g. GSK189254 and GSK239512, 5-HT4 receptor agonist, PPAR agonists, also NMDA receptor antagonists or modulators, also disease modifying agents such as α β or γ-secretase inhibitors (e.g. R-flurbiprofen), also AMPA positive modulators and Glycine Transporter Reuptake inhibitors.

When a compound of formula (I) or a pharmaceutically acceptable salt thereof is used in combination with another therapeutic agent, the compounds may be administered either sequentially or simultaneously by any convenient route.

The invention thus provides, in a further aspect, a combination comprising a compound of formula (I) or a pharmaceutically acceptable salt thereof together with a further therapeutic agent or agents.

A pharmaceutical composition of the invention, which may be prepared by admixture, suitably at ambient temperature and atmospheric pressure, is usually adapted for oral, parenteral or rectal administration and, as such, may be in the form of tablets, capsules, oral liquid preparations, powders, granules, lozenges, reconstitutable powders, injectable or infusable solutions or suspensions or suppositories. Orally administrable compositions are generally preferred.

Tablets and capsules for oral administration may be in unit dose form, and may contain conventional excipients, such as binding agents, fillers, tabletting lubricants, disintegrants and acceptable wetting agents. The tablets may be coated according to methods well known in normal pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain conventional additives such as suspending agents, emulsifying agents, non-aqueous vehicles (which may include edible oils), preservatives, and, if desired, conventional flavourings or colourants.

For parenteral administration, fluid unit dosage forms are prepared utilising a compound of the invention or pharmaceutically acceptable salt thereof and a sterile vehicle. The compound, depending on the vehicle and concentration used, can be either suspended or dissolved in the vehicle. In preparing solutions, the compound can be dissolved for injection and filter sterilised before filling into a suitable vial or ampoule and sealing. Advantageously, adjuvants such as a local anaesthetic, preservatives and buffering agents are dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilization cannot be accomplished by filtration. The compound can be sterilised by exposure to ethylene oxide before suspension in a sterile vehicle. Advantageously, a surfactant or wetting agent is included in the composition to facilitate uniform distribution of the compound.

The composition may contain from 0.1% to 99% by weight, preferably from 10% to 60% by weight, of the active material, depending on the method of administration. The dose of the compound of formula (I) as defined in the first and second aspect or a pharmaceutically acceptable salt thereof used in the treatment or prophylaxis of the aforementioned disorders will vary in the usual way with the seriousness of the disorders, the weight of the sufferer, and other similar factors. However, as a general guide suitable unit doses may be 0.05 to 1000 mg, more suitably 1.0 to 200 mg, and such unit doses may be administered more than once a day, for example two or three a day. Such therapy may extend for a number of weeks, months, years or even life.

A further aspect to the invention is a pharmaceutical composition comprising 0.05 to 1000 mg of a compound of formula (I) or a pharmaceutically acceptable salt thereof, and 0 to 3 g more suitably 0 to 2 g of at least one pharmaceutically acceptable carrier.

All publications, including but not limited to patents and patent applications, cited in this specification are herein incorporated by reference as if each individual publication were specifically and individually indicated to be incorporated by reference herein as though fully set forth.

### SUPPORTING COMPOUNDS

The preparation of a number of supporting compounds of formula (I) are described below.

### Intermediate 1

### 3-Chloro-5-(trifluoromethyl)benzenesulfonyl chloride

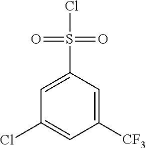 [SMILES: O=S(=O)(Cl)c1cc(Cl)cc(C(F)(F)F)c1]

A solution of NaNO₂ (6.50 g) was added to a suspension of 3-chloro-5-(trifluoromethyl)aniline (18.44 g) in concentrated HCl/acetic acid (83 mL/30 mL) at 0° C. and the mixture stirred for one hour. The diazonium salt formed was transferred into a saturated solution of sulfur dioxide in glacial acetic acid (350 mL) at 0° C. and the mixture was warmed up to room temperature for one hour. The mixture was poured onto ice-water and extracted with diethyl ether. The combined organic layers were washed with sodium hydrogen carbonate, dried over sodium sulphate, concentrated and purified by silica chromatography eluting with ethyl actetate/hexane to yield 3-chloro-5-(trifluoromethyl)benzenesulfonyl chloride (22 g, 84%).

¹H NMR (400 MHz, Chloroform-D) d ppm 8.0 (m, 1H) 8.2 (m, 1H) 8.2 (t, J=1.9 Hz, 1H).

### Compound 1

### 8-{[2-Chloro-4-(trifluoromethyl)phenyl]sulfonyl}-2,8-diazaspiro[4.5]decan-1-one (E1)

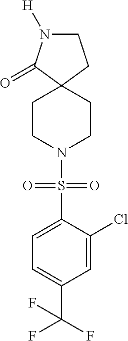 [SMILES: O=C1NCCC12CCN(S(=O)(=O)c1ccc(C(F)(F)F)cc1Cl)CC2]

2,8-Diazaspiro[4.5]decan-1-one hydrogen chloride (240 mg, 1.259 mmol) was dissolved in dichloromethane (10 mL) and triethylamine (0.526 mL, 3.78 mmol), and 2-chloro-4-(trifluoromethyl)benzenesulfonyl chloride (457 mg, 1.636 mmol) was added. After stirring for 16 h, the reaction mixture was washed sequentially with aqueous 0.5 M HCl and 0.5 M NaOH, the organic layer was passed through a hydrophobic frit, and concentrated in vacuo. The resulting residue was recrystallised from methanol to give 8-{[2-chloro-4-(trifluoromethyl)phenyl]sulfonyl}-2,8-diazaspiro[4.5]decan-1-one (300 mg, 0.718 mmol, 57% yield) as a white solid. 1H NMR (250 MHz, DMSO-d₆) δ ppm 1.38-1.49 (m, 2H) 1.65 (ddd, J=13.38, 10.87, 4.22 Hz, 2H) 1.91 (t, J=6.83 Hz, 2H) 2.96-3.09 (m, 2H) 3.14 (t, J=6.81 Hz, 2H) 3.65 (dt, J=12.99, 4.21 Hz, 2H) 7.61 (s, 1H) 7.94 (ddd, J=8.27, 1.82, 0.62 Hz, 1H) 8.14-8.22 (m, 2H). MS ES+ve m/z 397 (M+H).

### Compound 2

### 9-{[4-(Trifluoromethyl)phenyl]sulfonyl}-2,9-diazaspiro[5.5]undecan-1-one (E2)

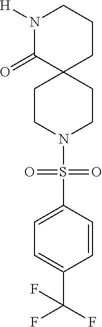 [SMILES: O=C1NCCCC12CCN(S(=O)(=O)c1ccc(C(F)(F)F)cc1)CC2]

2,9-Diazaspiro[5.5]undecan-1-one hydrogen chloride (115 mg, 0.562 mmol) was dissolved in dichloromethane (5 mL) and triethylamine (0.235 mL, 1.687 mmol), and 4-(trifluoromethyl)benzenesulfonyl chloride (179 mg, 0.731 mmol) was added. After stirring for 16 h, the reaction mixture was concentrated in vacuo and recrystallised from MeOH to give 9-{[4-(trifluoromethyl)phenyl]sulfonyl}-2,9-diazaspiro[5.5]undecan-1-one (80 mg, 0.210 mmol, 37% yield) as a white solid. 1H NMR (400 MHz, DMSO-d₆) δ ppm 1.41-1.54 (m, 4H) 1.55-1.63 (m, 2H) 1.92 (ddd, J=13.55, 9.17, 3.97 Hz, 2H) 2.82-2.91 (m, 2H) 3.04 (td, J=5.89, 2.19 Hz, 2H) 3.26-3.32 (m, 2H) 7.30 (br. s., 1H) 7.96 (d, J=8.17 Hz, 2H) 8.03 (d, J=8.33 Hz, 2H). MS ES+ve m/z 377 (M+H)

### Compound 3

### 8-{[4-(Trifluoromethyl)phenyl]sulfonyl}-2,8-diazaspiro[4.5]decan-1-one (E3)

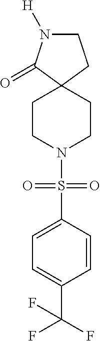 [SMILES: O=C1NCCC12CCN(S(=O)(=O)c1ccc(C(F)(F)F)cc1)CC2]

2,8-Diazaspiro[4.5]decan-1-one hydrogen chloride (280 mg, 1.469 mmol) was dissolved in dichloromethane (40 mL) and triethylamine (0.614 mL, 4.41 mmol), and 4-(trifluoromethyl)benzenesulfonyl chloride (467 mg, 1.909 mmol) was added. After 16 h, the reaction mixture was washed with aqueous 2 M HCl followed by aqueous 2 M NaOH, and the organic layer was passed through a hydrophobic frit and concentrated in vacuo. The resulting residue was purified by silica column chromatography on SP4 (gradient elution: 0-20% MeOH-DCM) to give 8-{[4-(trifluoromethyl)phenyl]sulfonyl}-2,8-diazaspiro[4.5]decan-1-one (532 mg, 1.453 mmol, 99% yield) as a white solid. 1H NMR (400 MHz, DMSO-d₆) δ ppm 1.45 (ddd, J=13.29, 3.51, 3.32 Hz, 2H) 1.61-1.72 (m, 2H) 1.77 (t, J=6.80 Hz, 2H) 2.60-2.70 (m, 2H) 3.09 (t, J=6.82 Hz, 2H) 3.49 (ddd, J=11.96, 4.65, 4.38 Hz, 2H) 7.60 (s, 1H) 7.97 (d, J=8.28 Hz, 2H) 8.04 (d, J=8.39 Hz, 2H). MS ES+ve m/z 363 (M+H).

### Compound 4

### 2-Methyl-8-{[4-(trifluoromethyl)phenyl]sulfonyl}-2,8-diazaspiro[4.5]decan-1-one (E4)

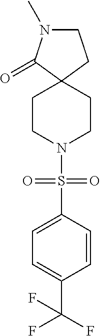 [SMILES: CN1CCC2(CCN(S(=O)(=O)c3ccc(C(F)(F)F)cc3)CC2)C1=O]

2-Methyl-2,8-diazaspiro[4.5]decan-1-one hydrogen chloride (128 mg, 0.624 mmol) was dissolved in dichloromethane (4 mL) and triethylamine (0.174 mL, 1.248 mmol), and DMAP (1 mg, 8.19 μmol) and 4-(trifluoromethyl)benzenesulfonyl chloride (168 mg, 0.687 mmol) were added. After stirring for 2 h the reaction mixture was concentrated in vacuo and the resulting residue was purified using MDAP to give 2-methyl-8-{[4-(trifluoromethyl)phenyl]sulfonyl}-2,8-diazaspiro[4.5]decan-1-one (176 mg, 0.458 mmol, 73% yield) as a white solid. 1H NMR (250 MHz, DMSO-d₆) δ ppm 1.38-1.51 (m, 2H) 1.58-1.77 (m, 4H) 2.60-2.74 (m, 5H) 3.15-3.23 (m, 2H) 3.43-3.54 (m, 2H) 7.97 (d, J=8.34 Hz, 2H) 8.04 (d, J=8.44 Hz, 2H). MS ES+ve m/z 377 (M+H).

### Compound 5

### 2-Methyl-8-{[3-(trifluoromethyl)phenyl]sulfonyl}-2,8-diazaspiro[4.5]decan-1-one (E5)

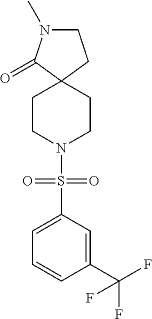 [SMILES: CN1CCC2(CCN(S(=O)(=O)c3cccc(C(F)(F)F)c3)CC2)C1=O]

2-Methyl-2,8-diazaspiro[4.5]decan-1-one hydrogen chloride (86 mg, 0.420 mmol) was dissolved in dichloromethane (5 mL) and triethylamine (0.129 mL, 0.924 mmol), before adding 3-(trifluoromethyl)benzenesulfonyl chloride (0.074 mL, 0.462 mmol). The reaction mixture was concentrated in vacuo and the resulting residue was purified using MDAP to give 2-methyl-8-{[3-(trifluoromethyl)phenyl]sulfonyl}-2,8-diazaspiro[4.5]decan-1-one (118 mg, 0.307 mmol, 73% yield) as a white solid. 1H NMR (400 MHz, DMSO-d₆) δ ppm 1.43 (t, J=3.75 Hz, 1H) 1.46 (t, J=3.84 Hz, 1H) 1.63-1.75 (m, 4H) 2.62-2.71 (m, 5H) 3.19 (t, J=6.91 Hz, 2H) 3.48 (t, J=4.93 Hz, 1H) 3.51 (t, J=4.96 Hz, 1H) 7.92 (t, J=7.86 Hz, 1H) 7.98 (s, 1H) 8.08 (d, J=7.95 Hz, 1H) 8.14 (d, J=7.84 Hz, 1H). MS ES+ve m/z 377 (M+H).

### Compound 6

### 2-Methyl-8-({4-[(trifluoromethyl)oxy]phenyl}sulfonyl)-2,8-diazaspiro[4.5]decan-1-one (E6)

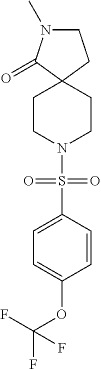 [SMILES: CN1CCC2(CCN(S(=O)(=O)c3ccc(OC(F)(F)F)cc3)CC2)C1=O]

2-Methyl-2,8-diazaspiro[4.5]decan-1-one hydrogen chloride (86 mg, 0.420 mmol) was dissolved in dichloromethane (5 mL) and triethylamine (0.129 mL, 0.924 mmol), before adding 4-[(trifluoromethyl)oxy]benzenesulfonyl chloride (0.071 mL, 0.420 mmol). The reaction mixture was concentrated in vacuo and the resulting residue was purified using MDAP to give 2-methyl-8-({4-[(trifluoromethyl)oxy]phenyl}sulfonyl)-2,8-diazaspiro[4.5]decan-1-one (103 mg, 0.257 mmol, 61% yield) as a white solid. 1H NMR (400 MHz, DMSO-d₆) δ ppm 1.43 (t, J=3.70 Hz, 1H) 1.46 (t, J=3.70 Hz, 1H) 1.62-1.75 (m, 4H) 2.59-2.68 (m, 5H) 3.18-3.22 (m, 2H) 3.45 (t, J=4.71 Hz, 1H) 3.48 (t, J=5.12 Hz, 1H) 7.64 (dd, J=8.85, 0.90 Hz, 2H) 7.87-7.92 (m, 2H). MS ES+ve m/z 393 (M+H).

### Compound 7

### 8-{[3-(Trifluoromethyl)phenyl]sulfonyl}-2,8-diazaspiro[4.5]decan-1-one (E7)

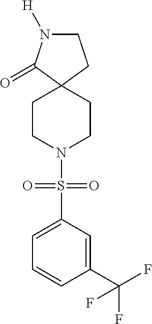 [SMILES: O=C1NCCC12CCN(S(=O)(=O)c1cccc(C(F)(F)F)c1)CC2]

2,8-Diazaspiro[4.5]decan-1-one hydrogen chloride (200 mg, 1.049 mmol) was dissolved in a mixture of dichloromethane (10 mL) and triethylamine (0.439 mL, 3.15 mmol), and 3-(trifluoromethyl)benzenesulfonyl chloride (308 mg, 1.259 mmol) was added. After 16 h, the reaction mixture was concentrated in vacuo, and the resulting residue was purified by silica column chromatography on SP4 (gradient elution: 0-20% MeOH-DCM) to give two batches of the desired product as white solids: 8-{[3-(trifluoromethyl)phenyl]sulfonyl}-2,8-diazaspiro[4.5]decan-1-one (204 mg, 0.557 mmol, 53% yield) and 8-{[3-(trifluoromethyl)phenyl]sulfonyl}-2,8-diazaspiro[4.5]decan-1-one (83 mg, 0.227 mmol, 22% yield). 1H NMR (400 MHz, DMSO-d₆) δ ppm 1.45 (ddd, J=13.47, 3.55, 3.43 Hz, 2H) 1.62-1.71 (m, 2H) 1.76 (t, J=6.80 Hz, 2H) 2.59-2.69 (m, 2H) 3.08 (t, J=6.82 Hz, 2H) 3.47-3.56 (m, 2H) 7.60 (s, 1H) 7.92 (t, J=7.87 Hz, 1H) 7.98 (br. s., 1H) 8.08 (d, J=8.00 Hz, 1H) 8.14 (d, J=7.78 Hz, 1H). MS ES+ve m/z 363 (M+H).

### Compound 8

### 8-({4-[(Trifluoromethyl)oxy]phenyl}sulfonyl)-2,8-diazaspiro[4.5]decan-1-one (E8)

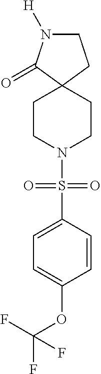 [SMILES: O=C1NCCC12CCN(S(=O)(=O)c1ccc(OC(F)(F)F)cc1)CC2]

2,8-Diazaspiro[4.5]decan-1-one hydrogen chloride (200 mg, 1.049 mmol) was dissolved in a mixture of dichloromethane (10 mL) and triethylamine (0.439 mL, 3.15 mmol), and 4-[(trifluoromethyl)oxy]benzenesulfonyl chloride (328 mg, 1.259 mmol) was added. After 3 h, the reaction mixture was concentrated in vacuo, and the resulting residue was purified by silica column chromatography on SP4 (gradient elution: 0-20% MeOH-DCM) to give 8-({4-[(trifluoromethyl)oxy]phenyl}sulfonyl)-2,8-diazaspiro[4.5]decan-1-one (300 mg, 0.769 mmol, 73% yield) as a white solid. 1H NMR (400 MHz, DMSO-d₆) δ ppm 1.45 (ddd, J=13.47, 3.49, 3.32 Hz, 2H) 1.62-1.71 (m, 2H) 1.76 (t, J=6.77 Hz, 2H) 2.57-2.65 (m, 2H) 3.09 (t, J=6.77 Hz, 2H) 3.43-3.51 (m, 2H) 7.60 (s, 1H) 7.64 (dd, J=8.91, 0.90 Hz, 2H) 7.87-7.92 (m, 2H). MS ES+ve m/z 379 (M+H).

### Compound 9

### 9-{[3-(Trifluoromethyl)phenyl]sulfonyl}-2,9-diazaspiro[5.5]undecan-1-one (E9)

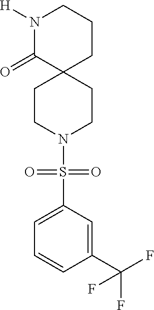 [SMILES: O=C1NCCCC12CCN(S(=O)(=O)c1cccc(C(F)(F)F)c1)CC2]

2,9-Diazaspiro[5.5]undecan-1-one hydrogen chloride (90 mg, 0.440 mmol) was dissolved in dichloromethane (10 mL) and triethylamine (0.123 mL, 0.879 mmol). 3-(Trifluoromethyl)benzenesulfonyl chloride (0.077 mL, 0.484 mmol) was added and stirred for 2 h. The mixture was then concentrated in vacuo and purified triturated with MeOH to give 9-{[3-(trifluoromethyl)phenyl]sulfonyl}-2,9-diazaspiro[5.5]undecan-1-one (11 mg, 0.028 mmol, 6% yield) as a white solid. 1H NMR (400 MHz, DMSO-d₆) δ ppm 1.40-1.53 (m, 4H) 1.55-1.63 (m, 2H) 1.87-1.96 (m, 2H) 2.87 (ddd, J=11.85, 8.81, 3.23 Hz, 2H) 3.04 (td, J=5.81, 1.70 Hz, 2H) 3.26-3.31 (m, 2H) 7.30 (br. s., 1H) 7.91 (t, J=7.92 Hz, 1H) 7.96 (s, 1H) 8.07 (d, J=7.78 Hz, 1H) 8.13 (d, J=7.84 Hz, 1H). MS ES+ve m/z 377 (M+H).

### Compound 10

### 9-({3-[(Trifluoromethyl)oxy]phenyl}sulfonyl)-2,9-diazaspiro[5.5]undecan-1-one (E10)

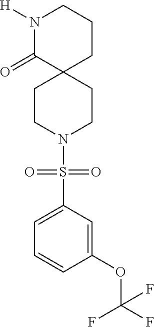 [SMILES: O=C1NCCCC12CCN(S(=O)(=O)c1cccc(OC(F)(F)F)c1)CC2]

2,9-Diazaspiro[5.5]undecan-1-one hydrogen chloride (107 mg, 0.524 mmol) was dissolved in dichloromethane (5 mL) and triethylamine (0.146 mL, 1.047 mmol). Then 3-[(trifluoromethyl)oxy]benzenesulfonyl chloride (0.098 mL, 0.577 mmol) was added and stirred for 16 h. The reaction mixture was concentrated in vacuo and the resulting residue was purified by MDAP to give 9-({3-[(trifluoromethyl)oxy]phenyl}-sulfonyl)-2,9-diazaspiro[5.5]undecan-1-one (73 mg, 0.184 mmol, 35% yield) as a white solid. 1H NMR (400 MHz, DMSO-d₆) δ ppm 1.37-1.54 (m, 4H) 1.54-1.64 (m, 2H) 1.86-1.96 (m, 2H) 2.86 (ddd, J=11.91, 8.78, 3.21 Hz, 2H) 3.04 (td, J=5.82, 2.00 Hz, 2H) 3.26-3.32 (m, 2H) 7.30 (br. s., 1H) 7.67 (s, 1H) 7.72-7.85 (m, 3H). MS ES+ve m/z 393 (M+H).

### Compound 11

### 2-Ethyl-8-{[4-(trifluoromethyl)phenyl]sulfonyl}-2,8-diazaspiro[4.5]decan-1-one (E11)

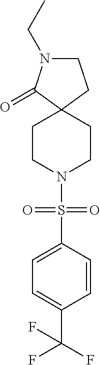 [SMILES: CCN1CCC2(CCN(S(=O)(=O)c3ccc(C(F)(F)F)cc3)CC2)C1=O]

To a suspension of 2-ethyl-2,8-diazaspiro[4.5]decan-1-one hydrogen chloride (200 mg, 0.914 mmol) in dichloromethane (8 mL) was added triethylamine (0.510 mL, 3.66 mmol) and 4-(trifluoromethyl)benzenesulfonyl chloride (268 mg, 1.097 mmol). The reaction mixture was stirred at room temperature under argon overnight (approximately 18 h). Water (40 mL) was added to the reaction mixture and stirred vigorously for 5 minutes, dichloromethane (50 mL×2) was used to wash the water and the organic phase was passed through a hydrophobic frit and evaporated. The crude material was purified by SP4, eluting with 100% iso-hexane for 3 column volumes then 0-100% EtOAc in iso-hexane over 20 column volumes. The product eluted at approx 70% EtOAc, the clean product containing fractions were collected and evaporated to give 2-ethyl-8-{[4-(trifluoromethyl)phenyl]sulfonyl}-2,8-diazaspiro[4.5]decan-1-one (185 mg, 0.474 mmol, 52% yield) as a white solid. 1H NMR (400 MHz, CHLOROFORM-d) δ ppm 1.08 (t, J=7.26 Hz, 3H) 1.55-1.61 (m, 2H) 1.82 (t, J=6.96 Hz, 2H) 1.91-2.01 (m, 2H) 2.96-3.08 (m, 2H) 3.21-3.32 (m, 4H) 3.48-3.56 (m, 2H) 7.80 (d, J=8.28 Hz, 2H) 7.90 (d, J=8.17 Hz, 2H). MS ES+ve m/z 391 (M+H).

### Compound 12

### 2-Ethyl-8-{[3-(trifluoromethyl)phenyl]sulfonyl}-2,8-diazaspiro[4.5]decan-1-one (E12)

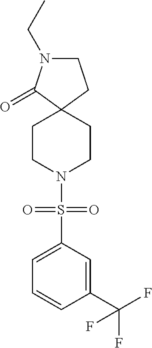 [SMILES: CCN1CCC2(CCN(S(=O)(=O)c3cccc(C(F)(F)F)c3)CC2)C1=O]

To a suspension of 2-ethyl-2,8-diazaspiro[4.5]decan-1-one hydrogen chloride (200 mg, 0.914 mmol) in dichloromethane (8 mL) was added triethylamine (0.510 mL, 3.66 mmol) and 3-(trifluoromethyl)benzenesulfonyl chloride (0.176 mL, 1.097 mmol). The reaction mixture was stirred at room temperature under argon overnight (approximately 18 h). Water (40 mL) was added to the reaction mixture and stirred vigorously for 5 minutes, dichloromethane (50 mL×2) was used to wash the water and the organic phase was passed through a hydrophobic frit and evaporated. The crude material was purified by SP4 eluting with 100% iso-hexane for 3 column volumes then 0-100% EtOAc in iso-hexane over 20 column volumes. The product eluted at approx 70% EtOAc, the clean product containing fractions were collected and evaporated, and freeze dried from 1,4-dioxane (3 mL) to give 2-ethyl-8-{[3-(trifluoromethyl)phenyl]sulfonyl}-2,8-diazaspiro[4.5]decan-1-one (209 mg, 0.535 mmol, 59% yield) as a white solid. 1H NMR (400 MHz, CHLOROFORM-d) δ ppm 1.07 (t, J=7.23 Hz, 3H) 1.58 (ddd, J=13.41, 7.52, 3.67 Hz, 2H) 1.81 (t, J=6.91 Hz, 2H) 1.90-2.02 (m, 2H) 3.00 (ddd, J=11.61, 8.13, 3.56 Hz, 2H) 3.22-3.32 (m, 4H) 3.52 (ddd, J=11.54, 7.54, 3.73 Hz, 2H) 7.69 (t, J=7.76 Hz, 1H) 7.86 (dd, J=7.84, 0.49 Hz, 1H) 7.96 (d, J=7.89 Hz, 1H) 8.04 (s, 1H). MS ES+ve m/z 391 (M+H).

### Compound 13

### 8-{[3-Fluoro-5-(trifluoromethyl)phenyl]sulfonyl}-2,8-diazaspiro[4.5]decan-1-one (E13)

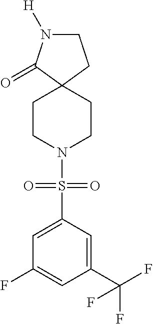 [SMILES: O=C1NCCC12CCN(S(=O)(=O)c1cc(F)cc(C(F)(F)F)c1)CC2]

2,8-Diazaspiro[4.5]decan-1-one hydrogen chloride (100 mg, 0.524 mmol) was dissolved in dichloromethane (10 mL) and triethylamine (0.219 mL, 1.573 mmol). Then 3-fluoro-5-(trifluoromethyl)benzenesulfonyl chloride (165 mg, 0.629 mmol) was added and stirred for 17 h. The mixture was concentrated in vacuo and the resulting residue was purified by MDAP to give two batches of product: 8-{[3-fluoro-5-(trifluoromethyl)phenyl]sulfonyl}-2,8-diazaspiro[4.5]decan-1-one (33.74 mg, 0.087 mmol, 17% yield) and 8-{[3-fluoro-5-(trifluoromethyl)phenyl]sulfonyl}-2,8-diazaspiro[4.5]decan-1-one (51 mg, 0.131 mmol, 25% yield) both as a white solid. 1H NMR (400 MHz, DMSO-d₆) δ ppm 1.41-1.48 (m, 2H) 1.61-1.70 (m, 2H) 1.80 (t, J=6.80 Hz, 2H) 2.64-2.73 (m, 2H) 3.10 (t, J=6.80 Hz, 2H) 3.55 (ddd, J=12.15, 4.34, 4.11 Hz, 2H) 7.61 (s, 1H) 7.84 (s, 1H) 8.00 (d, J=7.73 Hz, 1H) 8.18 (d, J=8.50 Hz, 1H). MS ES+ve m/z 381 (M+H).

### Compound 14

### 9-({4-[(Trifluoromethyl)oxy]phenyl}sulfonyl)-2,9-diazaspiro[5.5]undecan-1-one (E14)

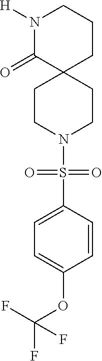 [SMILES: O=C1NCCCC12CCN(S(=O)(=O)c1ccc(OC(F)(F)F)cc1)CC2]

2,9-Diazaspiro[5.5]undecan-1-one hydrogen chloride (90 mg, 0.440 mmol) was dissolved in dichloromethane (10 mL) and triethylamine (0.123 mL, 0.879 mmol). 4-[(Trifluoromethyl)oxy]benzenesulfonyl chloride (0.082 mL, 0.484 mmol) was added and stirred for 2 h. The mixture was then concentrated in vacuo and triturated with MeOH to give 9-({4-[(trifluoromethyl)oxy]phenyl}sulfonyl)-2,9-diazaspiro[5.5]undecan-1-one (57 mg, 0.138 mmol, 31% yield) as a white solid. 1H NMR (400 MHz, DMSO-d₆) δ ppm 1.39-1.53 (m, 4H) 1.55-1.63 (m, 2H) 1.92 (ddd, J=13.54, 9.29, 3.81 Hz, 2H) 2.78-2.89 (m, 2H) 3.04 (td, J=5.52, 1.95 Hz, 2H) 3.28 (ddd, J=11.63, 7.11, 4.22 Hz, 2H) 7.30 (br. s., 1H) 7.63 (dd, J=8.80, 0.85 Hz, 2H) 7.88 (d, J=8.88 Hz, 2H). MS ES+ve m/z 393 (M+H).

### Compound 15

### 8-({3-[(Trifluoromethyl)oxy]phenyl}sulfonyl)-2,8-diazaspiro[4.5]decan-1-one (E15)

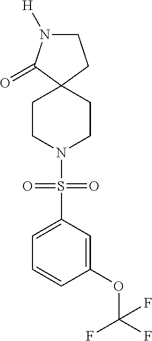 [SMILES: O=C1NCCC12CCN(S(=O)(=O)c1cccc(OC(F)(F)F)c1)CC2]

2,8-Diazaspiro[4.5]decan-1-one hydrogen chloride (100 mg, 0.524 mmol) was dissolved in dichloromethane (5 mL) and triethylamine (0.146 mL, 1.047 mmol). Then 3-[(trifluoromethyl)oxy]benzenesulfonyl chloride (0.098 mL, 0.577 mmol) was added and stirred for 16 h. The reaction mixture was concentrated in vacuo and the resulting residue was purified by MDAP to give 8-({3-[(trifluoromethyl)oxy]phenyl}-sulfonyl)-2,8-diazaspiro[4.5]decan-1-one (99 mg, 0.259 mmol, 49% yield) as a white solid. 1H NMR (400 MHz, DMSO-d₆) δ ppm 1.44 (ddd, J=13.41, 3.55, 3.32 Hz, 2H) 1.60-1.71 (m, 2H) 1.76 (t, J=6.80 Hz, 2H) 2.57-2.69 (m, 2H) 3.09 (t, J=6.82 Hz, 2H) 3.45-3.54 (m, 2H) 7.60 (s, 1H) 7.68 (dd, J=1.67, 0.79 Hz, 1H) 7.74-7.83 (m, 3H). MS ES+ve m/z 379 (M+H).

### Compound 16

### 9-{[3-Fluoro-5-(trifluoromethyl)phenyl]sulfonyl}-2,9-diazaspiro[5.5]undecan-1-one (E16)

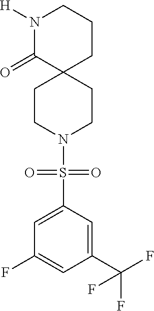 [SMILES: O=C1NCCCC12CCN(S(=O)(=O)c1cc(F)cc(C(F)(F)F)c1)CC2]

2,9-Diazaspiro[5.5]undecan-1-one hydrogen chloride (107 mg, 0.524 mmol) was dissolved in dichloromethane (10 mL) and triethylamine (0.219 mL, 1.573 mmol). Then 3-fluoro-5-(trifluoromethyl)benzenesulfonyl chloride (165 mg, 0.629 mmol) was added and stirred for 17 h. The mixture was concentrated in vacuo and the resulting residue was purified by MDAP to give 9-{[3-fluoro-5-(trifluoromethyl)phenyl]sulfonyl}-2,9-diazaspiro[5.5]undecan-1-one (91.67 mg, 0.228 mmol, 43% yield) as a white solid. 1H NMR (400 MHz, DMSO-d₆) δ ppm 1.45 (ddd, J=13.54, 5.92, 3.12 Hz, 2H) 1.51-1.65 (m, 4H) 1.92 (ddd, J=13.57, 9.40, 3.95 Hz, 2H) 2.82-2.95 (m, 2H) 3.00-3.09 (m, 2H) 3.34-3.40 (m, 2H) 7.32 (s, 1H) 7.83 (s, 1H) 7.98 (d, J=7.78 Hz, 1H) 8.17 (d, J=8.55 Hz, 1H). MS ES+ve m/z 395 (M+H).

### Compound 17

### 8-[(3,5-Dichlorophenyl)sulfonyl]-2,8-diazaspiro[4.5]decan-1-one (E17)

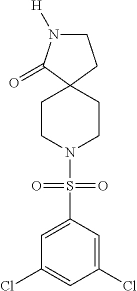 [SMILES: O=C1NCCC12CCN(S(=O)(=O)c1cc(Cl)cc(Cl)c1)CC2]

2,8-Diazaspiro[4.5]decan-1-one hydrogen chloride (111 mg, 0.584 mmol) was dissolved in dichloromethane (10 mL) and triethylamine (0.244 mL, 1.751 mmol), and 3,5-dichlorobenzenesulfonyl chloride (158 mg, 0.642 mmol) was added. After stirring for 17 h the reaction mixture was concentrated in vacuo and the resulting residue was purified by MDAP to give two batches of products: 8-[(3,5-dichlorophenyl)-sulfonyl]-2,8-diazaspiro[4.5]decan-1-one (53.5 mg, 0.144 mmol, 25% yield) and 8-[(3,5-dichlorophenyl)sulfonyl]-2,8-diazaspiro[4.5]decan-1-one (76.6 mg, 0.207 mmol, 35% yield) both as a white solid. 1H NMR (400 MHz, DMSO-d₆) δ ppm 1.45 (ddd, J=13.33, 3.27, 3.01 Hz, 2H) 1.60-1.72 (m, 2H) 1.81 (t, J=6.80 Hz, 2H) 2.61-2.74 (m, 2H) 3.10 (t, J=6.80 Hz, 2H) 3.52 (ddd, J=12.02, 4.48, 4.17 Hz, 2H) 7.61 (s, 1H) 7.76 (d, J=1.92 Hz, 2H) 8.05 (t, J=1.86 Hz, 1H). MS ES+ve m/z 363 (M+H).

### Compound 18

### 8-({3-Chloro-4-[(trifluoromethyl)oxy]phenyl}sulfonyl)-2,8-diazaspiro[4.5]decan-1-one (E18)

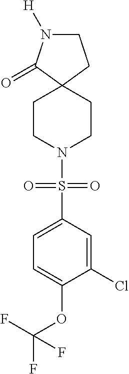 [SMILES: O=C1NCCC12CCN(S(=O)(=O)c1ccc(OC(F)(F)F)c(Cl)c1)CC2]

2,8-Diazaspiro[4.5]decan-1-one hydrogen chloride (185 mg, 0.973 mmol) was dissolved in a mixture of triethylamine (0.542 mL, 3.89 mmol) and dichloromethane (10 mL), and 3-chloro-4-[(trifluoromethyl)oxy]benzenesulfonyl chloride (344 mg, 1.167 mmol) was added. After 16 h the reaction mixture was concentrated in vacuo and the resulting residue was purified by silica column chromatography on SP4 (gradient elution: 0-20% MeOH-DCM) to give a yellow solid. The yellow solid was further purified on MDAP to give 8-({3-chloro-4-[(trifluoromethyl)oxy]phenyl}sulfonyl)-2,8-diazaspiro[4.5]decan-1-one (269 mg, 0.645 mmol, 66% yield) as a white solid. 1H NMR (400 MHz, DMSO-d₆) δ ppm 1.40-1.51 (m, 2H) 1.61-1.72 (m, 2H) 1.80 (t, J=6.80 Hz, 2H) 2.62-2.73 (m, 2H) 3.10 (t, J=6.82 Hz, 2H) 3.51 (ddd, J=12.00, 4.38, 4.17 Hz, 2H) 7.61 (s, 1H) 7.80-7.89 (m, 2H) 8.02-8.07 (m, 1H). MS ES+ve m/z 413 (M+H).

### Compound 19

### 2-(2,2,2-Trifluoroethyl)-8-{[4-(trifluoromethyl)phenyl]sulfonyl}-2,8-diazaspiro[4.5]decan-1-one (E19)

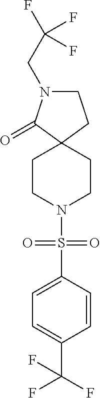 [SMILES: O=C1N(CC(F)(F)F)CCC12CCN(S(=O)(=O)c1ccc(C(F)(F)F)cc1)CC2]

8-{[4-(Trifluoromethyl)phenyl]sulfonyl}-2,8-diazaspiro[4.5]decan-1-one (308 mg, 0.850 mmol) was dissolved in DMF (8 mL) and cooled in an ice/water bath with stirring under argon. To this was added sodium hydride (54.4 mg, 1.360 mmol) and the suspension was stirred for 30 mins. 2,2,2-Trifluoroethyl trifluoromethanesulfonate (0.184 mL, 1.275 mmol) was added and the reaction was allowed to warm to room temperature and stirred for 1 h. The sample was reduced in vacuo. The residue was suspended in water (40 mL) and stirred vigorously for 5 minutes, dichloromethane (50 mL×2) was used to wash the water and the organic phase was passed through a hydrophobic frit and evaporated. The crude material was purified by SP4 eluting with 100% iso-hexane for 3 column volumes then 0-100% EtOAc in iso-hexane over 20 column volumes. The product peak streaked off the column over the 40-70% gradient. These fractions were collected and evaporated. The resulting solid was suspended in methanol (1 mL) and DMSO (1 mL) and purified by MDAP over two injections. The clean collected product peak was evaporated and freeze dried from 1,4-dioxane to give 2-(2,2,2-trifluoroethyl)-8-{[4-(trifluoromethyl)phenyl]sulfonyl}-2,8-diazaspiro[4.5]decan-1-one (60 mg, 0.135 mmol, 16% yield) as a white solid. 1H NMR (400 MHz, CHLOROFORM-d) δ ppm 1.60-1.70 (m, 2H) 1.91 (t, J=6.91 Hz, 2H) 1.97 (ddd, J=13.65, 7.65, 3.59 Hz, 2H) 3.09 (ddd, J=11.63, 7.66, 3.78 Hz, 2H) 3.37-3.53 (m, 4H) 3.84 (q, J=9.08 Hz, 2H) 7.81 (d, J=8.28 Hz, 2H) 7.91 (d, J=8.17 Hz, 2H). MS ES+ve m/z 445 (M+H).

### Compound 20

### 8-{[3-Methyl-5-(trifluoromethyl)phenyl]sulfonyl}-2,8-diazaspiro[4.5]decan-1-one (E20)

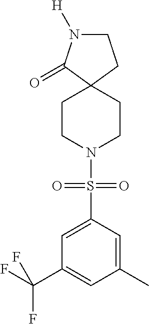 [SMILES: Cc1cc(C(F)(F)F)cc(S(=O)(=O)N2CCC3(CCNC3=O)CC2)c1]

2,8-Diazaspiro[4.5]decan-1-one hydrogen chloride (200 mg, 1.049 mmol) was dissolved in a mixture of triethylamine (0.439 mL, 3.15 mmol) and dichloromethane (10 mL), and 3-bromo-5-(trifluoromethyl)benzenesulfonyl chloride (407 mg, 1.259 mmol) was added. The reaction mixture was stirred for 16 h and the reaction mixture was concentrated in vacuo. The resulting yellow solid 8-{[3-bromo-5-(trifluoromethyl)phenyl]sulfonyl}-2,8-diazaspiro[4.5]decan-1-one (819 mg, impure) was used in the next reaction without further purification. MS ES+ve m/z 443 (M+H). 8-{[3-Bromo-5-(trifluoromethyl)phenyl]sulfonyl}-2,8-diazaspiro[4.5]decan-1-one (819 mg, impure) and potassium carbonate (217 mg, 1.574 mmol) was suspended in 1,4-dioxane (10 mL). Trimethylboroxine (0.219 mL, 1.574 mmol) and Pd(PPh₃)₄ (121 mg, 0.105 mmol) were then added and the reaction mixture was heated to 100° C. After 20 h, the reaction was cooled, filtered through a hydrophobic frit, and concentrated in vacuo. The resulting residue was purified by silica column chromatography on SP4 (gradient elution: 0-20% MeOH-DCM). The resulting brown residue was further purified on MDAP to give 8-{[3-methyl-5-(trifluoromethyl)phenyl]sulfonyl}-2,8-diazaspiro[4.5]decan-1-one (126 mg, 0.331 mmol, 32% yield) as a white solid. 1H NMR (400 MHz, DMSO-d₆) δ ppm 1.46 (dt, J=13.41, 3.46 Hz, 2H) 1.61-1.72 (m, 2H) 1.77 (t, J=6.80 Hz, 2H) 2.52 (s, 3H) 2.57-2.66 (m, 2H) 3.09 (t, J=6.80 Hz, 2H) 3.46-3.54 (m, 2H) 7.55-7.63 (m, 1H) 7.76 (s, 1H) 7.89 (s, 1H) 7.96 (s, 1H). MS ES+ve m/z 377 (M+H).

### Compound 21

### 8-{[2-Methyl-4-(trifluoromethyl)phenyl]sulfonyl}-2,8-diazaspiro[4.5]decan-1-one (E21)

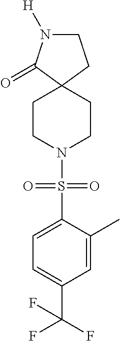 [SMILES: Cc1cc(C(F)(F)F)ccc1S(=O)(=O)N1CCC2(CCNC2=O)CC1]

2,8-Diazaspiro[4.5]decan-1-one hydrogen chloride (200 mg, 1.049 mmol) was dissolved in a mixture of triethylamine (0.439 mL, 3.15 mmol) and dichloromethane (10 mL), and 2-bromo-4-(trifluoromethyl)benzenesulfonyl chloride (407 mg, 1.259 mmol) was added. The reaction mixture was stirred for 16 h and the reaction mixture was concentrated in vacuo. The resulting yellow solid 8-{[2-bromo-4-(trifluoromethyl)phenyl]sulfonyl}-2,8-diazaspiro[4.5]decan-1-one (820 mg, impure) was used in the next reaction without further purification. MS ES+ve m/z 443 (M+H). 8-{[2-Bromo-4-(trifluoromethyl)phenyl]sulfonyl}-2,8-diazaspiro[4.5]decan-1-one (820 mg, impure) and potassium carbonate (217 mg, 1.574 mmol) was suspended in 1,4-dioxane (10 mL). Trimethylboroxine (0.219 mL, 1.574 mmol) and Pd(PPh₃)₄ (121 mg, 0.105 mmol) were then added and the reaction mixture was heated to 100° C. After 20 h, the reaction was cooled, filtered through a hydrophobic frit, and concentrated in vacuo. The resulting residue was purified by silica column chromatography on SP4 (gradient elution: 0-20% MeOH-DCM). The resulting brown residue was further purified on MDAP to give 8-{[2-methyl-4-(trifluoromethyl)phenyl]sulfonyl}-2,8-diazaspiro[4.5]decan-1-one (61 mg, 0.160 mmol, 15% yield) as a white solid. 1H NMR (400 MHz, DMSO-d₆) δ ppm 1.39-1.49 (m, 2H) 1.59-1.69 (m, 2H) 1.89 (t, J=6.80 Hz, 2H) 2.64 (s, 3H) 2.85-2.96 (m, 2H) 3.13 (t, J=6.82 Hz, 2H) 3.54 (ddd, J=12.48, 4.01, 3.84 Hz, 2H) 7.63 (s, 1H) 7.79 (d, J=7.67 Hz, 1H) 7.89 (s, 1H) 8.01 (d, J=8.22 Hz, 1H). MS ES+ve m/z 377 (M+H).

### Compound 22

### 8-{[2-Methyl-5-(trifluoromethyl)phenyl]sulfonyl}-2,8-diazaspiro[4.5]decan-1-one (E22)

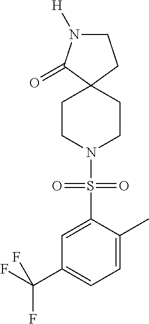 [SMILES: Cc1ccc(C(F)(F)F)cc1S(=O)(=O)N1CCC2(CCNC2=O)CC1]

2,8-Diazaspiro[4.5]decan-1-one hydrogen chloride (200 mg, 1.049 mmol) was dissolved in a mixture of triethylamine (0.439 ml, 3.15 mmol) and dichloromethane (10 ml), and 2-bromo-5-(trifluoromethyl)benzenesulfonyl chloride (407 mg, 1.259 mmol) was added. The reaction mixture was stirred for 16 h and the reaction mixture was concentrated in vacuo. The resulting yellow solid 8-{[2-bromo-5-(trifluoromethyl)phenyl]sulfonyl}-2,8-diazaspiro[4.5]decan-1-one (825 mg, impure) was used in the next reaction without further purification. MS ES+ve m/z 443 (M+H). 8-{[2-bromo-5-(trifluoromethyl)phenyl]sulfonyl}-2,8-diazaspiro[4.5]decan-1-one (825 mg, impure) and potassium carbonate (217 mg, 1.574 mmol) was suspended in 1,4-dioxane (10 mL). Trimethylboroxine (0.219 mL, 1.574 mmol) and Pd(PPh₃)₄ (121 mg, 0.105 mmol) were then added and the reaction mixture was heated to 100° C. After 20 h, the reaction was cooled, filtered through a hydrophobic frit, and concentrated in vacuo. The resulting residue was purified by silica column chromatography on SP4 (gradient elution: 0-20% MeOH-DCM). The resulting brown residue was further purified on MDAP twice to give 8-{[2-methyl-5-(trifluoromethyl)phenyl]sulfonyl}-2,8-diazaspiro[4.5]decan-1-one (41 mg, 0.107 mmol) as a white solid. 1H NMR (400 MHz, DMSO-d₆) δ ppm 1.39-1.49 (m, 2H) 1.58-1.70 (m, 2H) 1.89 (t, J=6.80 Hz, 2H) 2.65 (s, 3H) 2.84-2.95 (m, 2H) 3.13 (t, J=6.80 Hz, 2H) 3.54 (ddd, J=12.63, 4.08, 3.95 Hz, 2H) 7.63 (s, 1H) 7.74 (d, J=7.95 Hz, 1H) 7.98 (dd, J=8.03, 1.40 Hz, 1H) 8.03 (d, J=1.21 Hz, 1H). MS ES+ve m/z 377 (M+H).

### Compounds 23-27

The compounds of Table 1 were prepared by a similar procedure to that described for compound 4 using the appropriate amine and sulphonyl chloride.

TABLE 1

| | | | | Reten- |
|---|---|---|---|---|
| Com- | | | m/z | tion |
| pound | | | [M + | Time |
| no. | Compound name | Structure | H]⁺ | (min) |
| 23 | 8-{[3-Chloro-5- (trifluoromethyl) phenyl]sulfonyl}- 2,8-diazaspiro [4.5]decan-1- one (E23) | 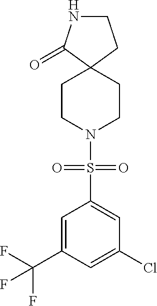 [SMILES: O=C1NCCC12CCN(S(=O)(=O)c1cc(Cl)cc(C(F)(F)F)c1)CC2] | 397 399 | 1.04 |
| 24 | 9-({3- [(Difluoromethyl) oxy]phenyl} sulfonyl)-2,9- diazaspiro[5.5] undecan-1-one (E24) | 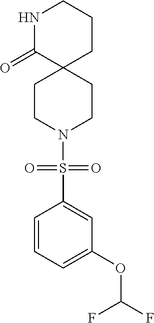 [SMILES: O=C1NCCCC12CCN(S(=O)(=O)c1cccc(OC(F)F)c1)CC2] | 375 | 0.91 |
| 25 | 8-{[3,5- Bis(trifluoro- methyl)phenyl] sulfonyl}-2,8- diazaspiro[4.5] decan-1-one (E25) | 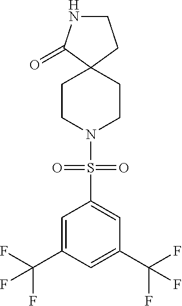 [SMILES: O=C1NCCC12CCN(S(=O)(=O)c1cc(C(F)(F)F)cc(C(F)(F)F)c1)CC2] | 431 | 1.08 |
| 26 | 8-({3- [(Difluoromethyl) oxy]phenyl} sulfonyl)-2,8- diazaspiro[4.5] decan-1-one (E26) | 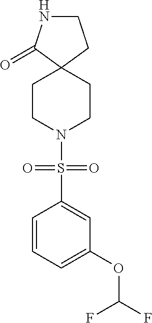 [SMILES: O=C1NCCC12CCN(S(=O)(=O)c1cccc(OC(F)F)c1)CC2] | 361 | 0.85 |
| 27 | 9-{[2,5- Bis(trifluoro- methyl)phenyl] sulfonyl}-2,9- diazaspiro[5.5] undecan-1-one (E27) | 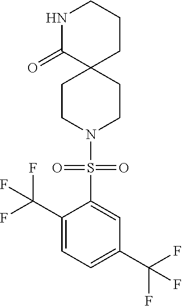 [SMILES: O=C1NCCCC12CCN(S(=O)(=O)c1cc(C(F)(F)F)ccc1C(F)(F)F)CC2] | 445 | 1.07 |

### Abbreviations

| | |
|---|---|
| DCM | Dichloromethane |
| DMF | N,N-dimethylformamide |
| MeOH | Methanol |
| DMSO | N,N-Dimethylsulfoxide |
| LiHMDS | Lithium hexamethyldisilazide |
| TMS | Trimethylsilyl |
| DMAP | 4-(Dimethylamino)pyridine |
| h | Hours |
| EtOAc | Ethyl acetate |
| NMR | Nuclear magnetic resonance |
| | (spectroscopy) |
| ppm | Parts per million |
| δ | Chemical shift |
| DMSO-d₆ | Deuterated dimethyl sulfoxide |
| CHLOROFORM-d | Deuterated chloroform |
| TFA | Trifluoroacetic acid |
| LCMS | Liquid chromatography-Mass |
| | spectroscopy |
| MDAP | Mass directed automated preparation |

### Equipment

### ¹H NMR Spectra

Chemical shifts are expressed in parts per million (ppm, units). Coupling constants (J) are in units of hertz (Hz). Splitting patterns describe apparent multiplicities and are designated as s (singlet), d (doublet), t (triplet) q (quartet), dd (double doublet), dt (double triplet), m (multiplet), br (broad).

### Mass-Directed Automated HPLC/Mass-Directed Automated Preparation (MDAP)

Where indicated in the above compounds, purification by mass-directed automated HPLC was carried out using the following apparatus and conditions:

### Hardware

Waters 2525 Binary Gradient Module
Waters 515 Makeup Pump
Waters Pump Control Module
Waters 2767 Inject Collect
Waters Column Fluidics Manager
Waters 2996 Photodiode Array Detector
Waters ZQ Mass Spectrometer
Gilson 202 fraction collector
Gilson Aspec waste collector

### Software

Waters Mass Lynx version 4 SP2

### Column

The columns used are Waters Atlantis, the dimensions of which are 19 mm×100 mm (small scale) and 30 mm×100 mm (large scale). The stationary phase particle size is 5 μm.

### Solvents

A: Aqueous solvent=Water+0.1% Formic Acid
B: Organic solvent=Acetonitrile+0.1% Formic Acid
Make up solvent=Methanol:Water 80:20
Needle rinse solvent=Methanol

### Methods

There are five methods used depending on the analytical retention time of the compound of interest. They have a 13.5-minute runtime, which comprises of a 10-minute gradient followed by a 3.5 minute column flush and re-equilibration step.

### Large/Small Scale 1.0-1.5=5-30% B

### Large/Small Scale 1.5-2.2=15-55% B

### Large/Small Scale 2.2-2.9=30-85% B

### Large/Small Scale 2.9-3.6=50-99% B

Large/Small Scale 3.6-5.0=80-99% B (in 6 minutes followed by 7.5 minutes flush and re-equilibration)

### Flow Rate

All of the above methods have a flow rate of either 20 mL/min (Small Scale) or 40 mL/min (Large Scale).

### Shallow Gradients

Large 1.5 to 2.3 min=13-29% B
Large 1.9 to 2.3 min=25-41% B
Large 2.3 to 2.6 min=37-53% B
Large 2.6 to 3.1 min=49-65% B
Large 3.1 to 3.6 min=61-77% B

### Liquid Chromatography/Mass Spectrometry

Analysis of the above compounds by Liquid Chromatography/Mass Spectrometry (LC/MS) was carried out using the following apparatus and conditions:

### Hardware

### Waters Acquity Binary Solvent Manager

### Waters Acquity Sample Manager

### Waters Acquity PDA

### Waters ZQ Mass Spectrometer

### Sedere Sedex 75

### Software

Waters MassLynx version 4.1

### Column

The column used is a Waters Acquity BEH UPLC C18, the dimensions of which are 2.1 mm×50 mm. The stationary phase particle size is 1.7 μm.

### Solvents

A: Aqueous solvent=Water+0.05% Formic Acid
B: Organic solvent=Acetonitrile+0.05% Formic Acid

### Weak Wash=1:1 Methanol:Water

### Strong Wash=Water

### Method

The generic method used has a 2 minute runtime.

| Time/min | % B |
|---|---|
| 0 | 3 |
| 0.1 | 3 |
| 1.5 | 97 |
| 1.9 | 97 |
| 2.0 | 3 |

The above method has a flow rate of 1 ml/min.

The injection volume for the generic method is 0.5 μl

The column temperature is 40 deg The UV detection range is from 220 to 330 nm

### Biotage SP4®

Biotage-SP40 is an automated purification system. It uses preloaded silica gel columns. The user applies their material to the top of the column and chooses solvents, gradients, flow rates, column size, collection method and eluting volumes.

### Phase Separators (Hydrophobic Frit)

Phase separators are a range of ISOLUTE® columns fitted with an optimized frit material that easily separates aqueous phase from chlorinated solvents under gravity.

### SCX—Strong Cation Exchange Cartridge

Where indicated in the Compounds, an SCX cartridge was used as part of the compound purification process. Typically an ISOLUTE SCX-2 cartridge was used. ISOLUTE SCX-2 is a silica-based sorbent with a chemically bonded propylsulfonic acid functional group.

### ISOLUTE SCX-2 Chemical Data

### Base Material: Silica, 50 μm

Functional Group: Propylsulfonic acid
Capacity: 0.6 meq/g

### Counter Ion: Proton

### ISOLUTE NH2—Weak Anion Exchange Cartridge

Where indicated in the compounds, an isolute NH2 cartridge was used as part of the compound purification process. Typically an ISOLUTE NH2 cartridge was used. ISOLUTE NH2 is a silica-based sorbent with a chemically bonded aminopropyl functional group.

Description: Aminopropyl functionalized silica. Manufactured using trifunctional silane. pK 9.8. Non end-capped.

### Average Particle Size: 50 μm

### Nominal Porosity: 60 Å

Exchange Capacity: 0.6 meq/g
Comments: Weak anion exchange sorbent for extraction of strongly ionized acidic drugs, particularly for ease of elution.

### Pharmacological Data

Compounds of the invention may be tested for in vitro biological activity in the hCaᵥ2.2 assay in accordance with the following studies:

### Methods

### Cell Biology

Stable cell lines expressing the human Caᵥ2.2 α (α1_{B}) subunit, along with the human β3 and α2δ1 auxiliary subunits were created following sequential transfection and selection of human embryonic kidney (HEK293) cells. HEK293 cells were cultured in Dulbecco's modified Eagles media/F12 media (Invitrogen, Cat #041-95750V) containing 10% fetal bovine serum, with added L-glutamine (2 mM; Invitrogen, Cat #25030-024) and non-essential amino acids (5%; Invitrogen, Cat #11140-035). Initially HEK293 cells were transfected with two plasmid vectors for expression of the hCaᵥ2.2 α subunit (pCIN5-hCaᵥ2.2 which carries a neomycin resistance marker) and the hCaᵥ β3 subunit (pCIH-hCaᵥ β3 which carries a hygromycin resistance marker). Clonal cell lines were isolated following selection in media supplemented with 0.4 mg ml⁻¹ Geneticin G418 (Invitrogen, Cat #10131-027) and 0.1 mg ml⁻¹ hygromycin (Invitrogen, Cat #10687-010). These clonal cell lines were assessed for Caᵥ2.2 α/β3-mediated current expression using the IonWorks planar array electrophysiology technology (described below). A clonal line was identified that gave a reasonable level of functional Caᵥ2.2 α/β3 current expression. This cell line was transfected with a plasmid vector for expression of the human α2δ1 subunit (pCIP-α2δ1 which carries a puromycin resistance marker) and clonal cell lines isolated following selection in media containing 0.62 μg ml⁻¹ puromycin (Sigma, Cat #P-7255), in addition to 0.4 mg ml⁻¹ Geneticin G418 and 0.1 mg ml⁻¹ hygromycin. Several cell lines were identified that gave robust levels of Caᵥ2.2 α/β3/α2δ1-mediated current expression and one of these was selected for compound profiling. Expression of all three subunits within this cell line was continuously maintained by the inclusion of G418 (0.4 mg ml⁻¹), hygromycin (0.1 mg ml⁻¹) and puromycin (0.62 μg ml⁻¹). Cells were maintained at 37° C. in a humidified environment containing 5% CO₂ in air. Cells were liberated from the T175 culture flasks for passage and harvesting using TrpLE (Invitrogen, Cat #12604-013).

### Cell Preparation

Cells were grown to 30-60% confluence in T175 flasks and maintained at 30° C. for 24 hrs prior to recording. Cells were lifted by removing the growth media, washing with Ca²⁺ free PBS (Invitrogen, Cat #14190-094) and incubating with 3 ml of warmed (37° C.) TrpLE (Invitrogen, Cat #12604-013) for 6 minutes. Lifted cells were suspended in 10 ml of extracellular buffer. Cell suspension was then placed into a 15 ml tube and centrifuged for 2 minutes at 700 rpm. After centrifugation, the supernatant was removed and the cell pellet was resuspended in 4.5 ml of extracellular solution.

### Electrophysiology

Currents were recorded at room temperature (21-23° C.) using the IonWorks planar array electrophysiology technology (Molecular Devices Corp.). Stimulation protocols and data acquisition were carried out using a microcomputer (Dell Pentium 4). In order to determine planar electrode hole resistances (Rp), a 10 mV, 160 ms potential difference was applied across each hole. These measurements were performed before cell addition. After cell addition a seal test was performed prior to antibiotic (amphotericin) circulation to achieve intracellular access. Leak subtraction was conducted in all experiments by applying a 160 ms hyperpolarizing (10 mV) prepulse 200 ms before the test pulses to measure leak conductance. Test pulses stepping from the holding potential (V_{H}) of −90 mV to +10 mV were applied for 20 ms and repeated 10 times at a frequency of 10 Hz. In all experiments, the test pulse protocol was performed in the absence (pre-read) and presence (post-read) of a compound. Pre- and post-reads were separated by a compound addition followed by a 3-3.5 min incubation.

### Solutions and Drugs

The intracellular solution contained the following (in mM): K-gluconate 120, KCl 20 mM, MgCl₂ 5, EGTA 5, HEPES 10, adjusted to pH 7.3. Amphotericin was prepared as 30 mg/ml stock solution and diluted to a final working concentration of 0.2 mg ml⁻¹ in intracellular buffer solution. The extracellular solution contained the following (in mM): Na-gluconate 120, NaCl 20, MgCl₂ 1, HEPES 10, BaCl₂ 5, adjusted to pH 7.4.

Compounds were prepared in DMSO as 10 mM stock solutions and subsequent 1:3 serial dilutions performed. Finally the compounds were diluted 1:100 in external solution resulting in a final DMSO concentration of 1%.

### Data Analysis

The recordings were analysed and filtered using seal resistance (>40 MΩ), resistance reduction (>35%) and peak current amplitude (>200 pA) in the absence of compound to eliminate unsuitable cells from further analysis. Paired comparisons between pre-compound and post-compound additions were used to determine the inhibitory effect of each compound. The concentrations of compounds required to inhibit current elicited by the 1^{st} depolarising pulse by 50% (tonic pIC50) were determined by fitting of the Hill equation to the concentration response data. In addition the use-dependent inhibitory properties of the compounds were determined by assessing the effect of compounds on the 10^{th} versus 1^{st} depolarising pulse. The ratio of the 10^{th} over 1^{st} pulse was determined in the absence and presence of drug and the % use-dependent inhibition calculated. The data was fitted using the same equation as for the tonic pIC₅₀ and the concentration producing 30% inhibition (use-dependent pUD₃₀) determined.

The compounds 1 to 27 were tested in the hCaᵥ2.2 assay.

The compounds 1 to 27 exhibited a pUD₃₀ value of 4.5 or more than 4.5. The compounds 1 to 5, 7, 9 to 24 exhibited a pUD₃₀ value of 5.0 or more than 5.0. The compounds 9, 10 and 20 exhibited a pUD₃₀ value of 5.5 or more than 5.5.

The compounds 1 to 27 exhibited a mean pIC₅₀ value of 4.5 or less than 4.5.

1. A compound of formula (I), or a salt thereof: 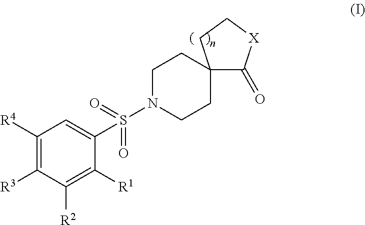
wherein R¹ and R⁴ are independently selected from hydrogen, chlorine, bromine, methyl, trifluoromethyl or trifluoromethoxy;
R² represents hydrogen, chlorine, fluorine, bromine, methyl, trifluoromethyl, difluoromethoxy or trifluoromethoxy;
R³ represents hydrogen, chlorine, bromine, trifluoromethyl or trifluoromethoxy;
such that at least one of R¹, R², R³ and R⁴ represents a group other than hydrogen and such that when one of R¹, R², R³ or R⁴ represents methyl, at least one other of R¹, R², R³ or R⁴ represents a group other than hydrogen and such that when R² represents fluorine, R⁴ represents trifluoromethyl and such that when R³ represents trifluoromethyl, R² represents a group other than chlorine;
n represents an integer from 1 or 2;
X represents —N—(R⁵)—; and
R⁵ represents hydrogen or C₁₋₄ alkyl optionally substituted by one or more chlorine or fluorine atoms.

2. A compound as defined in claim 1, wherein n represents 1.

3. A compound as defined in claim 1, wherein R¹, R² and R⁴ each represent hydrogen and R³ represents trifluoromethyl or trifluoromethoxy, such as trifluoromethyl.

4. A compound as defined in claim 1, wherein R¹, R³ and R⁴ each represent hydrogen and R² represents trifluoromethyl, difluoromethoxy or trifluoromethoxy.

5. A compound as defined in claim 1, wherein R² and R⁴ each represent hydrogen, R¹ represents methyl or chlorine and R³ represents trifluoromethyl.

6. A compound as defined in claim 1, wherein R¹ and R³ each represent hydrogen, R² represents fluorine, chlorine, methyl or trifluoromethyl and R⁴ represents chlorine or trifluoromethyl.

7. A compound as defined in claim 1, wherein R¹ and R⁴ each represent hydrogen, R² represents chlorine and R³ represents trifluoromethoxy.

8. A compound as defined in claim 1, wherein R² and R³ each represent hydrogen, R¹ represents methyl and trifluoromethyl and R⁴ represents trifluoromethyl.

9. A compound as defined in claim 1, wherein R¹ and R³ each represent hydrogen, R² represents fluorine and R⁴ represents trifluoromethyl.

10. A compound as defined in claim 1, wherein R⁵ represents hydrogen or C₁₋₄ alkyl, such as methyl or ethyl, optionally substituted by one or more chlorine or fluorine atoms, such as trifluoroethane.

11. A compound as defined in claim 10, wherein R⁵ represents hydrogen.

12. A compound or salt as defined in claim 1 which is selected from:
8-{[2-Chloro-4-(trifluoromethyl)phenyl]sulfonyl}-2,8-diazaspiro[4.5]decan-1-one (E1);
9-{[4-(Trifluoromethyl)phenyl]sulfonyl}-2,9-diazaspiro[5.5]undecan-1-one (E2);
8-{[4-(Trifluoromethyl)phenyl]sulfonyl}-2,8-diazaspiro[4.5]decan-1-one (E3);
2-Methyl-8-{[4-(trifluoromethyl)phenyl]sulfonyl}-2,8-diazaspiro[4.5]decan-1-one (E4);
2-Methyl-8-{[3-(trifluoromethyl)phenyl]sulfonyl}-2,8-diazaspiro[4.5]decan-1-one (E5);
2-Methyl-8-({4-[(trifluoromethyl)oxy]phenyl}sulfonyl)-2,8-diazaspiro[4.5]decan-1-one (E6);
8-{[3-(Trifluoromethyl)phenyl]sulfonyl}-2,8-diazaspiro[4.5]decan-1-one (E7);
8-({4-[(Trifluoromethyl)oxy]phenyl}sulfonyl)-2,8-diazaspiro[4.5]decan-1-one (E8);
9-{[3-(Trifluoromethyl)phenyl]sulfonyl}-2,9-diazaspiro[5.5]undecan-1-one (E9);
9-({3-[(Trifluoromethyl)oxy]phenyl}sulfonyl)-2,9-diazaspiro[5.5]undecan-1-one (E10);
2-Ethyl-8-{[4-(trifluoromethyl)phenyl]sulfonyl}-2,8-diazaspiro[4.5]decan-1-one (E11);
2-Ethyl-8-{[3-(trifluoromethyl)phenyl]sulfonyl}-2,8-diazaspiro[4.5]decan-1-one (E12);
8-{[3-Fluoro-5-(trifluoromethyl)phenyl]sulfonyl}-2,8-diazaspiro[4.5]decan-1-one (E13);
9-({4-[(Trifluoromethyl)oxy]phenyl}sulfonyl)-2,9-diazaspiro[5.5]undecan-1-one (E14);
8-({3-[(Trifluoromethyl)oxy]phenyl}sulfonyl)-2,8-diazaspiro[4.5]decan-1-one (E15);
9-{[3-Fluoro-5-(trifluoromethyl)phenyl]sulfonyl}-2,9-diazaspiro[5.5]undecan-1-one (E16);
8-[(3,5-Dichlorophenyl)sulfonyl]-2,8-diazaspiro[4.5]decan-1-one (E17);
8-({3-Chloro-4-[(trifluoromethyl)oxy]phenyl}sulfonyl)-2,8-diazaspiro[4.5]decan-1-one (E18);
2-(2,22-Trifluoroethyl)-8-{[4-(trifluoromethyl)phenyl]sulfonyl}-2,8-diazaspiro[4.5]decan-1-one (E19);
8-{[3-Methyl-5-(trifluoromethyl)phenyl]sulfonyl}-2,8-diazaspiro[4.5]decan-1-one (E20);
8-{[2-Methyl-4-(trifluoromethyl)phenyl]sulfonyl}-2,8-diazaspiro[4.5]decan-1-one (E21);
8-{[2-Methyl-5-(trifluoromethyl)phenyl]sulfonyl}-2,8-diazaspiro[4.5]decan-1-one (E22);
8-{[3-Chloro-5-(trifluoromethyl)phenyl]sulfonyl}-2,8-diazaspiro[4.5]decan-1-one (E23);
9-({3-[(Difluoromethyl)oxy]phenyl}sulfonyl)-2,9-diazaspiro[5.5]undecan-1-one (E24);
8-{[3,5-Bis(trifluoromethyl)phenyl]sulfonyl}-2,8-diazaspiro[4.5]decan-1-one (E25);
8-({3-[(Difluoromethyl)oxy]phenyl}sulfonyl)-2,8-diazaspiro[4.5]decan-1-one (E26); and
9-{[2,5-Bis(trifluoromethyl)phenyl]sulfonyl}-2,9-diazaspiro[5.5]undecan-1-one (E27).

13. A compound or salt as defined in claim 12 which is 8-{[4-(Trifluoromethyl)phenyl]sulfonyl}-2,8-diazaspiro[4.5]decan-1-one (E3).

14. A pharmaceutical composition which comprises a compound as defined in claim 1, or a pharmaceutically acceptable salt thereof, and a pharmaceutically acceptable carrier or excipient.

15. A compound, or pharmaceutically acceptable salt thereof, as defined in claim 1 for use in therapy.

16. A compound as defined in claim 1 for use in the treatment of pain.

17. A process for the preparation of a compound of formula (I) or a salt thereof as defined in claim 1, which process comprises:
(a) reacting a compound of formula (II) 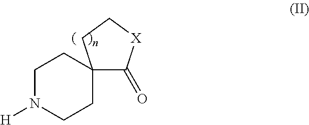
or a protected derivative thereof, wherein X and n are as defined in claim 1, with a compound of formula (III) 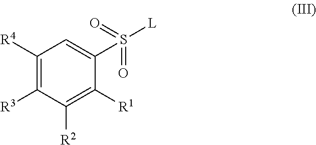
wherein R¹, R², R³ and R⁴ are as defined in claim 1 and L¹ represents a suitable leaving group such as a halogen atom, such as chlorine;
(b) deprotecting a compound of formula (I) or converting groups which are protected; and optionally thereafter
(c) interconversion to other compounds of formula (I).
